(19) United States
(12) Patent Application Publication
(10) Pub. No.: US 2007/0269376 A1
(43) Pub. Date: Nov. 22, 2007

(54) Method for Determining Dosage for an Oral Killed Vaccine

(75) Inventors: Robert Clancy, North South Wales (AU); Phillip Comans, North South Wales (AU); Gerald Pang, North South Wales (AU)

Correspondence Address: FULBRIGHT & JAWORSKI L.L.P, 2200 ROSS AVENUE, SUITE 2800, DALLAS, TX, 75201-2784, US

(73) Assignee: Hunter Immunology Limited, Newcastle (AU)

(21) Appl. No.: 11/573,890

(22) PCT Filed: Aug. 17, 2005
(86) PCT No.: PCT/AU05/01229

§ 371 Date: Jul. 23, 2007

(30) Foreign Application Priority Data
Aug. 17, 2004 (AU) 2004904671

(51) Int. Cl. A61K 39/02; A61K 39/00; A61K 39/085; A61K 39/104; A61K 39/108; A61K 39/118; A61P 31/00; A61P 37/00

(52) U.S. Cl. 424/9.1; 424/184.1; 424/234.1; 424/244.1; 424/256.1; 424/257.1; 424/260.1; 424/263.1; 424/274.1

(57) ABSTRACT

There is disclosed a method for determining an administration regimen for an oral killed vaccine for use in immunising individuals in a population against an infection or disease. The method comprise administering the oral killed vaccine to one or more individuals in a population and identifying an indicative dosaging level of the vaccine which induces a reduction in immune system responsiveness to the vaccine in the one or more individuals. A further dosaging level that elicits an immune response in one or more individuals of the population without inducing the reduction in immune system responsiveness to the vaccine is then determined.

### FIELD OF THE INVENTION

The present invention relates to a method for determining an administration regimen for an oral killed vaccine suitable for use in immunising against an infection or disease.

### BACKGROUND OF THE INVENTION

Anti-bacterial vaccines are known in the art and examples include Haemophilus influenzae B vaccine which consists of bacterial polysaccharide conjugated with tetanus toxoid protein. Killed bacterial vaccines for the prophylaxis or treatment of enteric infections have also been known for some time and a killed bacterial vaccine for typhoid fever is commercially available. These vaccines are predominantly if not exclusively administered by injection and serve as “classic” vaccines in that they aim to stimulate a systemic antibody response to provide protection against disease.

An oral killed bacterial vaccine against infection by non-typable Haemophilus influenzae (NTHi) is also known in the art. NTHi is the bacteria most commonly linked with nasal and bronchus colonisation in subjects with chronic lung disease, and has been linked to acute episodes of bronchitis in these subjects. A significant factor in the generation of acute bronchitis in such subjects is the uncontrolled and inappropriate migration of neutrophils into the bronchus lumen in response to the colonising bacteria. The accumulation of neutrophil-laden fluid within the bronchi results in purulent sputum. The use of the oral NTHi killed bacterial vaccine has been shown to protect against purulent sputum production, high levels of bacterial colonisation of the airways and environmental spread of the bacteria as assessed by acquisition of infection by bystander subjects. This vaccine stimulates the common mucosal system following activation of gut-associated lymphoid tissue (GALT) and more specifically, Peyer's patches in the intestines.

Antigen administered orally is processed by GALT differently from systemic lymphoid tissue. Teleologically, this can be understood in terms of mucosal physiology where environmental “antigen” needs to be excluded but not at the cost of damaging mucosal “inflammation”. A powerful suppression mechanism therefore exists, to minimise potentially damaging immune responses to such antigen. This concept was originally identified as “split tolerance” where a systemic immune response (ie. mediated by the generation of antibody) was associated with the failure to detect a mucosal antibody response (tolerance). Research using orally administered killed influenza virus shows that an antibody response is stimulated over a narrow range of antigen dose. This immunisation “zone” is flanked by low and high “zone” tolerance. The same concept applies to cellular immunity though the zone in which T-lymphocyte-mediated responses may be stimulated appears to be marginally wider, with protection occurring without an antibody response. The outcome of antigen interaction with GALT is the selective migration of B and T-lymphocytes to distant mucosal sites of infection where they mediate protection. However, while the NTHi vaccine proved of clinical value, the level of protection afforded against mucosal infection by NTHi in different individuals as judged by the reduction and the number and degree of acute episodes of bronchitis is variable.

### SUMMARY OF THE INVENTION

Broadly stated, the present invention relates to a method for determining an administration regimen for an oral killed vaccine based on identification of an indicative dosaging level which induces switching of the immune system from a state of responsiveness to the vaccine to a state of tolerance. The variation in mucosal immunity in an outbred population associated with the use of oral killed vaccines in the past is believed to arise at least in part, from the use of less than optimal administration regimen. By determining an indicative dosaging level for an outbred population at which the switching over to a state of tolerance is induced for a given oral killed vaccine, an optimised administration regimen can be identified for generating immunity in different individuals within the population.

More particularly, in a first aspect of the present invention there is provided a method for determining an administration regimen for an oral killed vaccine, comprising:
administering the oral killed vaccine to one or more individuals in a population;
identifying an indicative dosaging level of the vaccine which induces a reduction in immune system responsiveness to the vaccine in the one or more individuals; and
determining a further dosaging level that elicits an immune response in one or more individuals of the population without inducing the reduction in immune system responsiveness to the vaccine.

Typically, the oral killed vaccine will be administered to a plurality of individuals, and an indicative dosaging level of the vaccine which induces the reduction in the immune system responsiveness in all or at least a majority of the individuals will be identified.

The indicative dosaging level may comprise a single dosage of the oral killed vaccine, or a course of administration comprising a plurality of dosages of the oral killed vaccine which may be the same or different. When a course of administration of the vaccine is utilised, the interval between each dosage may vary. The further dosaging level can be derived by modifying the indicative dosage level. Modification of the indicative dosage level may for instance, comprise one or more of lowering the, or each, dosage of the vaccine, reducing or increasing the number of dosages of the vaccine administered or the number of courses of administration of the vaccine, and varying (eg increasing) the interval or intervals between dosages.

Preferably, the further dosaging level will be selected such that substantially maximal induction of the immune response by the indicative dosage level is achieved by the vaccine substantially without inducing the reduction in the immune system responsiveness to the vaccine.

In another aspect of the present invention there is provided a method for formulating a dosage regimen for an oral killed vaccine, comprising:
determining a dosaging level of the vaccine that generates an immune response in one or more individuals of a population below an indicative dosaging level of the vaccine that induces a reduction in immune system responsiveness to the vaccine in one or more individuals of the population, the dosaging level which generates the immune response being selected to achieve substantially maximal induction of the immune response.

Immune system responsiveness to the vaccine can be determined by measuring one or more parameters associated with activation of antigen responsive cells by the vaccine. The antigen responsive cells will normally comprise one or more of antigen presenting cells, and B- and/or T-lymphocytes. Preferably, the cells will comprise one or both of antigen presenting cells and T-lymphocytes. The antigen presenting cells will typically comprise macrophages. Most preferably, the T-lymphocytes will comprise Th1 cells. Activation of the antigen responsive cells is to be taken in its broadest sense to encompass direct and/or indirect activation of the cells. By “direct” activation is meant the vaccine activates at least some of the antigen responsive cells by contact with them such as when antigen of the vaccine is bound or phagocytosed by the cells. By “indirect” activation is meant at least some of the antigen responsive cells are activated by interaction with cells such as macrophages that have contacted antigen of the vaccine or for instance, by cytokine(s) or other chemical messenger(s) the release of which has been elicited or induced by the vaccine, or a combination of such possibilities.

Typically, the oral killed vaccine will be a vaccine against abnormal or undesirable colonisation of a mucosal surface of the individual such as by a bacteria, fungi or yeast. Preferably, the vaccine will be an oral killed bacterial vaccine. Most preferably, the vaccine will comprise one or more whole killed microbial organisms. However, the invention is not limited to the use of whole killed organisms and methods described herein also apply to oral killed vaccines comprising soluble and/or particulate matter derived from microbial organisms.

Typically also, the immune response elicited by the vaccine will predominantly if not substantially exclusively, comprise a cellular immune response.

In another aspect, there is provided a method for immunising an individual with an oral killed vaccine, comprising:
administering an effective amount of the vaccine to the individual utilising an administration regimen for the vaccine that has been determined by a method according to the first aspect of the invention.

The mammal may be any mammal treatable with an oral killed vaccine, such as a primate, a member of the rodent family such as a rat or mouse, or a member of the bovine, porcine, ovine or equine families. Preferable, however, the mammal will be a human being.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

The features and advantages of the present invention will become further apparent from the following description of preferred embodiments.

### BRIEF DESCRIPTION OF THE FIGURES

FIG. 1 is a graph showing the proliferative response of T-lymphocytes from human subjects with brochiectasis, chronic cough and purulent sputum, treated with three courses of different dosages of a soluble Ps. aeruginosa antigen over an 84 day evaluation period.

FIG. 2 is a graph showing the proliferative response of T-lymphocytes from the subjects to the non-specific T-cell mitogen phytohemagglutinin (PHA) over the evaluation period.

FIG. 3 is a graph showing the variation in the level of sputum purulence in the subjects over the evaluation period.

FIG. 4 is a graph showing variation in sputum bacteria count in the subjects over the evaluation period.

### DETAILED DESCRIPTION OF THE PREFERRED EMBODIMENTS

Methods embodied by the present invention find particular application in determining administration regimen for oral killed vaccines for the treatment or prophylaxis of microbial infections of lung and other respiratory tract mucosal surfaces, as well as other mucosal sites in the body such as the oral, nasal, oropharyngeal, pharyngeal, digestive, vaginal, eye associated and urinary mucosal surfaces. Bacteria contained in oral killed vaccines employed in methods of the invention may for instance be selected from Chlamydia species, Haemophilus species, Non-typable Haemophilus influenzae (NTHi) species, Pseudomonas species, Streptococcus species, Staphylococcus species, E. coli species, Mycoplasma species and Helicobacter species amongst others. The vaccines may also incorporate combinations of different species of such bacteria or other microbial organisms. Microbial organisms other than bacteria that may be utilised in such vaccines include Candida species, (eg Candida albicans) and yeast species such as Saccharomyces species.

Oral killed bacterial vaccines that may be administered in accordance with an administration regimen determined by a method of the invention include oral killed vaccines against infections selected from the group consisting of NTHi, S. aureus, Ps. aeruginosa, S. pneumoniae, and combinations thereof. P. aeruginosa for instance can colonise not only the respiratory tract but can also infect eye mucosa and the ear cavity. NTHi has also been implicated in a range of infectious conditions including otitis media and in the exacerbation of pneumonia and chronic bronchitis. Accordingly, vaccines containing one or more killed isolates of these bacteria may be administered for the prophylaxis or treatment of such associated conditions. By way of further examples, vaccines comprising killed Non-typable H. influenzae, S. pneumoniae or P. aeruginosa may be utilised in the prophylaxis or treatment of bronchitis or pneumonia, acute infections in cystic fibrosis and chronic obstructive airways disease, sinus disease, and compromised lung function as well as other lung and respiratory tract diseases and disorders.

Preferred parameters indicative of the level of activation of antigen responsive cells such as one or both of macrophages and T-lymphocytes, comprise cellular proliferation and particularly T-lymphocyte proliferation, cell surface antigen expression, measurement of cell effector function(s), and cytokine production. The antigen responsive cells can be isolated from lymph ducts and/or blood of individuals for characterisation of such parameters.

Cellular proliferation may be conveniently evaluated by cell counts, ³H-thymidine uptake and/or MTT assays. Cell surface antigen expression of antigen responsive cells can also be readily determined by flow cytometric analysis involving labelling cell surface antigens known to be up regulated or down regulated as a result of cellular activation, utilising appropriately labelled antibodies specific for such surface antigens. For example, activated T-lymphocytes express up regulated levels of lymphocyte function-associated antigen-1(LFA-1), CD2, CTLA-4, IL-2 receptor, CD4, T-cell receptor, L-selectin, CD40 ligand and CD45RO. An example of a cell surface molecule that is down regulated with activation of T-lymphocytes is CD45RA. Similarly, activated antigen presenting cells express up regulated levels of CD80, CD86, MHC II molecules, CD14, CD11c and CD18.

Cytokine expression may be measured directly by capture or sandwich enzyme linked immunosorbent assays (ELISA), or indirectly by cell growth assays in which the cytokine of interest acts as a growth factor or inhibitor. Cytokine expression may also be evaluated by determining the level of expression of mRNA coding for the cytokine by employing reverse transcriptase polymerase chain reaction (RT-PCR), or by in-situ hybridisation protocols utilising single cells or cell populations and specific oligonucleotide probes as is known in the art.

IL-12 is produced by antigen presenting cells in the early stages of activation and in combination with γ-IFN, induces proliferating CD4+ T-lymphocytes to differentiate into Th1 cells. Th1 cells stimulate infected macrophages through secretion of γ-IFN and interaction of the CD40 ligand expressed by the Th1 cells with the CD40 receptor expressed by macrophages. More broadly, Th1 cells stimulate the antibacterial mechanisms of phagocytic cells (eg neutrophils and macrophages) and release cytokines that attract such phagocytic cells to sites of infection. Besides IFN-γ, Th1 cells typically also secrete IL-12 and TNF-β.

While both Th1 and Th2 cells secrete IL-3, GM-CSF and for instance TNF-α, the overall cytokine profiles for Th1 and Th2 cells are different. More particularly, activation of Th2 cells results predominantly in a humoral immune response characterised by the activation of B-lymphocytes and the generation of antibodies by the activated B cells, while Th1 cells mediate a non-antibody cellular immune response. Cytokines characteristic of Th2 cell driven immune response include IL-4, IL-5, IL-10, IL-13 and TGF-β. Hence, measurement of the level of the secretion of for instance, one or both of IL-12 and γ-IFN is useful for assessment of the state of activation of antigen-presenting cells and/or Th1 committed CD4⁺ T-lymphocytes.

An indicative dosaging level of an oral killed vaccine under consideration at which switching over of the immune system to a state of tolerance is induced can be identified by administering a course of the vaccine known to induce an immune response to a group of individuals, repeating the course of administration a number of times, and measuring the level of activation of antigen responsive cells from recipients over the evaluation period. The course of vaccine administration may for instance, comprise a single dose of the vaccine or daily administration of the vaccine for two or more days. The course of administration can for example be repeated at an interval of from about 2 weeks up to about 6 weeks and more preferably, at an interval of from about 3 weeks to 5 weeks each time. Induction of non-responsiveness is indicated by a sustained reduction in the activation state of the antigen responsive cells from a maximal level of immune response to the vaccine. An optimised dosaging level of the vaccine that does not result in the reduction in the immune system responsiveness to the vaccine can then be identified, such as by increasing the interval between courses of the vaccine with or without increasing the or each course of administration (eg. up to 10-14 days in length) or for instance, by selecting a lower dosage of the vaccine and maintaining the same interval(s) between administration of each course.

Alternatively, different dosages of the vaccine can be administered to different groups of individuals within a population, and the highest dosage at which the reduction in the immune response to the vaccine occurs identified. An optimised dosaging level may then be obtained by selecting a lower dosage of the vaccine which generates an effective or substantially maximal immune response without inducing switching over of the immune response to a state of non-responsiveness. The population will generally be a normal population and the groups of individuals will typically be essentially representative of the population. The groups may comprise random groups of individuals or for instance, be representative of a given age or weight range within the population.

Vaccines administered in accordance with the invention will typically comprise the selected bacterial isolate(s) in an amount of between about 5% to about 80% w/w of the vaccine composition. The dosage of the, or each, bacterial isolate administered will typically be in a range of from about 10⁹ to about 10¹², more preferably from about 10¹⁰ to about 10¹¹ cfu, respectively.

The vaccine itself may be freeze-dried or lyophilised for later reconstitution utilising a physiologically acceptable buffer or fluid. The vaccine can also contain one or more anti-caking agents, isotonic agents, preservatives such as thimerosal, stabilisers such as amino acids and sugar moieties, sweetening agents such sucrose, lactose or saccharin, pH modifiers sodium hydroxide, hydrochloric acid, monosodium phosphate and/or disodium phosphate, a pharmaceutically acceptable carrier such as physiologically saline, suitable buffers, solvents, dispersion media and isotonic preparations. Use of such ingredients and media for pharmaceutically active substances and vaccines is well known in the art. Supplementary active agents such as one or more cytokines for boosting the immune response, particularly cytokines characteristic of a Th1 response such as γ-IFN, IL-12 and TNF-β, may also be incorporated in the vaccine. A vaccine may also comprise one or more adjuvants. Adjuvants, pharmaceutically acceptable carriers and combinations of ingredients that may be utilised in oral killed vaccines can for instance be found in handbooks and texts well known to the skilled addressee such as “Remington: The Science and Practice of Pharmacy (Mack Publishing Co., 1995)”, the contents of which is incorporated herein in its entirety by reference.

The oral killed bacterial vaccine may be administered as a dry powder or in liquid form. Administration can for example be achieved by aerosol inhalation, as a dosed liquid, by instillation, or as a spray. Devices for facilitating for delivery of oral vaccines are well known in the art and include metered dose inhalers (MDIs), dry powder inhalers (DPIs) and nebulisers including those which use ultrasonic energy or compressed air or other propellant to achieve atomisation. Propellants which may be used in MDIs include for instance chlorofluorocarbons (CFCs) such as trichlorofluorocarbon (CFC-11) and dichlorodifluorocarbon (CFC-12) and hydrofluoroalkanes.

### EXAMPLE 1

### Identification of an Optimal Dose of Killed Ps. aeruginosa for Use as an Oral Vaccine

In this study, nine human subjects with bronchiectasis and chronic cough and purulent sputum, were given a killed oral bacterial vaccine against Ps. aeruginosa (Ps. a) infection in three courses—at day 0, 28, and 56. Each course comprised the oral administration of two tablets per day, for three consecutive days. Each tablet contained 10¹¹ killed whole Ps. a bacteria. T-lymphocytes were separated from blood, stimulated with a soluble Ps. a and proliferation was detected by measurement of H³-thymidine uptake.

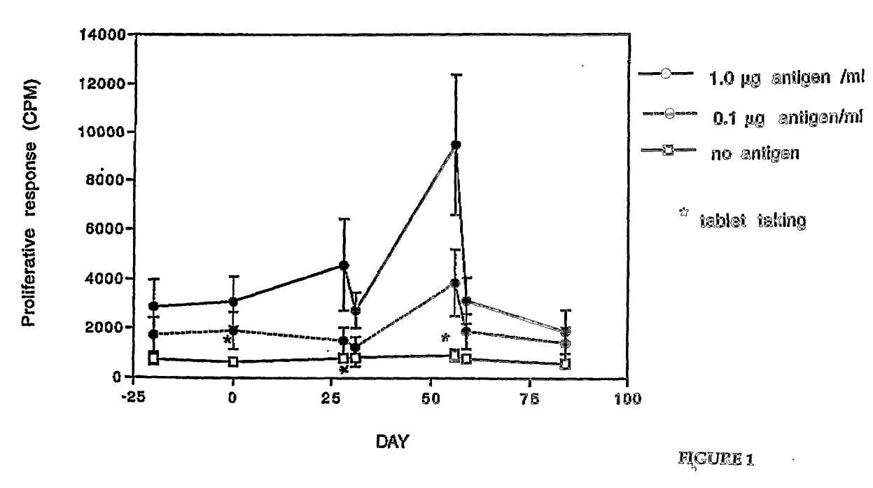

Following course (1) at day 28 and course (2) at day 56, an increase in uptake of H³-thymidine was noted (see FIG. 1). The fall in response three days after each course (due to temporary sequestration in gut-associated lymphoid tissue) was followed by an increase in circulating sensitised T-lymphocytes for the first (2) courses. However, at day 84, circulating T-lymphocyte were non-responsive to the added antigen, reflecting the induction of a state of non-responsiveness. This indicates that the immunisation regimen was not optimised.

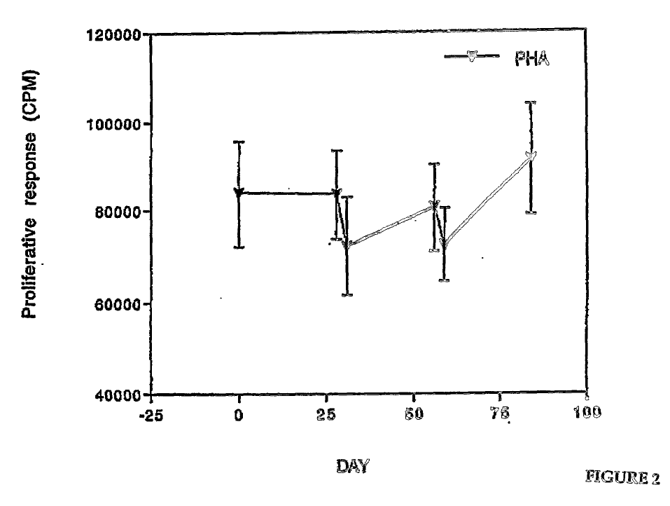

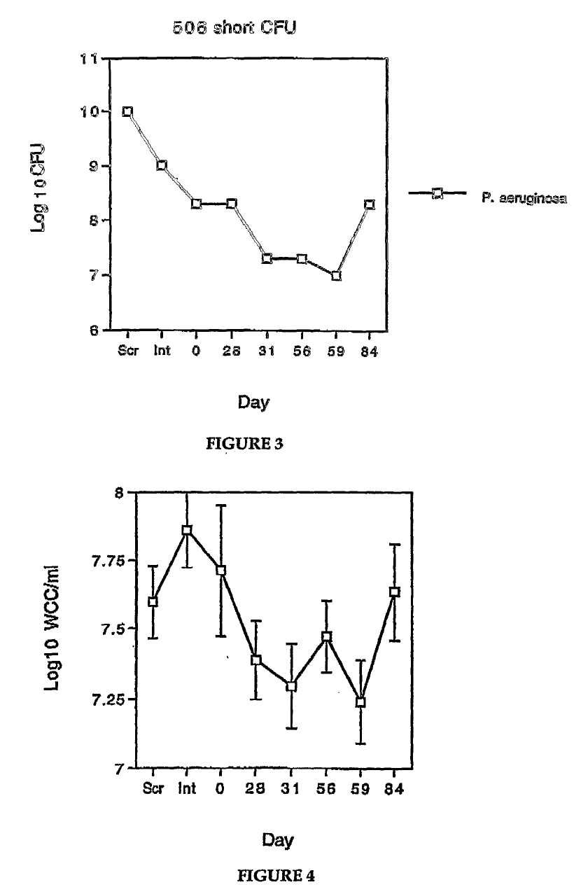

Optimisation can be achieved by reducing the dose and/or by altering the schedule such as by restricting the administration of the vaccine to two courses, or by maintaining administration of three dosages of the vaccine but reducing the final dose of the vaccine. That these results reflect a specific down regulation of Ps. a-related immunity can be seen by retention of responsiveness to the non-specific T cell mitogen, phytohemagglutinin (PHA) (see FIG. 2) over the 84 day period. That this day 84 down regulation reflects a loss of vaccine-induced immunity compared to the two previous courses, is shown by the increase in sputum purulence measured at day 84, compared to the first (ie. day 28) and second (ie. day 56) oral courses (see FIG. 3). A similar increase in sputum bacteria count between day 56 and day 84 was observed (ie. following course 2 and 3, respectively) with an increase in bacteria count in subjects 1 to 5 and 8. No change in bacteria counts were observed in subject 2 while a slight fall was observed in subject 1 (see FIG. 4). All the results are shown as mean values with associated standard errors (S.E.).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for determining an administration regimen for an oral killed vaccine, comprising:
administering the oral killed vaccine to one or more individuals in a population;
identifying an indicative dosaging level of the vaccine which induces a reduction in immune system responsiveness to the vaccine in the one or more individuals; and
determining a further dosaging level that elicits an immune response in one or more individuals of the population without inducing the reduction in immune system responsiveness to the vaccine.

2. A method according to claim 1 wherein the vaccine is administered to a plurality of individuals in the population, and an indicative dosage level of the vaccine which induces the reduction in the immune system responsiveness in all or a majority of the individuals is identified.

3. A method according to claim 1 wherein the indicative dosaging level comprises a single dosage of the vaccine, or a course of administration comprising a plurality of dosages of the vaccine which are the same or different.

4. A method according to claim 1 wherein the further dosaging level is derived by modifying the indicative dosaging level.

5. A method according to claim 4 wherein the further dosaging level is derived by modifying the indicative dosage level involving employing one or more modifications selected from the group consisting of lowering a dosage or dosages of the vaccine, reducing or increasing a course or courses of administration of the vaccine, and varying an interval or intervals between courses of the vaccine.

6. A method according to claim 1 wherein the further dosaging level is selected such that substantially maximal induction of the immune response by the indicative dosage level is achieved by the vaccine without inducing the reduction in the immune system responsiveness to the vaccine.

7. A method according to claim 1 wherein the immune system responsiveness to the vaccine is determined by measuring one or more parameters associated with activation of antigen responsive cells by the vaccine.

8. A method according to claim 7 wherein the antigen responsive cells comprise one or both of antigen presenting cells and lymphocytes.

9. A method according to claim 8 wherein the antigen presenting cells comprise macrophages.

10. A method according to claim 8 wherein the lymphocytes comprise T-lymphocytes.

11. A method according to claim 7 wherein the one or more parameters associated with activation of the antigen responsive cells are selected from the group consisting of cellular proliferation, cell surface antigen expression, measurement of one or more cell effector functions, and cytokine production.

12. A method according to claim 8 comprising measuring at least one parameter indicative of antigen presenting cell activation level and at least one further parameter indicative of T-lymphocyte activation level.

13. A method according to claim 12 wherein the parameter indicative of antigen presenting cell activation level comprises IL-12 expression.

14. A method according to claim 12 wherein the parameter indicative of T-lymphocyte activation level comprises □-IFN expression.

15. A method according to claim 1 wherein the immune response comprises predominantly a cellular immune response.

16. A method according to claim 1 wherein the vaccine comprises one or more whole killed microbial organisms, and/or soluble and/or particulate matter thereof.

17. A method according to claim 1 wherein the oral killed vaccine comprises a vaccine against abnormal or undesirable colonisation of a mucosal surface by a microbial organism selected from the group consisting of bacteria, fungi and yeast.

18. A method according to claim 17 wherein the microbial organism is selected from the group consisting of Chlamydia species, Haemophilus species, Non-typable Haemophilus influenzae species, Pseudomonas species, Streptococcus species, Staphylococcus species, E. coli species, Mycoplasma species, Helicobacter species, Candida species and Saccharomyces species.

19. A method according to claim 1 wherein the oral killed vaccine is an oral killed bacterial vaccine.

20. A method according to claim 19 wherein the microbial organism is selected from the group consisting of Non-typable H. influenzae, S. pneumoniae, P. aeruginosa and S. aureus.

21. A method for immunizing an individual with an oral killed vaccine, the method comprising:
administering an effective amount of the vaccine to the individual utilising an administration regimen for the vaccine that has been determined by a method as defined in any one of claims 1 to 20.

22. A method for formulating a dosage regimen for an oral killed vaccine, comprising:
determining a dosaging level of the vaccine that generates an immune response in one or more individuals of a population below an indicative dosaging level of the vaccine that induces a reduction in immune system responsiveness to the vaccine in one or more individuals of the population, the dosaging level which generates the immune response being selected to achieve substantially maximal induction of the immune response.

23. A method according to claim 22 wherein the reduction in the immune system responsiveness is reflected in one or more parameters associated with activation of antigen responsive cells by the vaccine.

24. A method according to claim 23 wherein the antigen responsive cells comprise one or both of antigen presenting cells and lymphocytes.

25. (canceled)

26. A method according to claim 24 wherein the lymphocytes comprise T-lymphocytes.

27. A method according to claim 23 wherein the one or more parameters are selected from the group consisting of cellular proliferation, cell surface antigen expression, measurement of one or more cell effector functions, and cytokine production.

28. A method according to claim 23 wherein the one or more parameters comprise at least one parameter indicative of antigen presenting cell activation level and at least one further parameter indicative of T-lymphocyte activation level.

29.-34. (canceled)

35. A method according to claim 22 wherein the oral killed vaccine is an oral killed bacterial vaccine.

36. A method according to claim 35 wherein the microbial organism is selected from the group consisting of Non-typable H. influenzae, S. pneumoniae, P. aeruginosa and S. aureus.
